(19) United States
(12) Patent Application Publication
(10) Pub. No.: US 2006/0122117 A1
(43) Pub. Date: Jun. 8, 2006

(54) Method of treating retinopathies and disorders associated with blood vessel loss

(75) Inventor: Lois E.H. Smith, West Newton, MA (US)

Correspondence Address: David S. Resnick, Esq.;NIXON PEABODY LLP, 100 Summer Street, Boston, MA, 02110, US

(73) Assignee: Children's Medical Center Corporation, Boston, MA (US)

(21) Appl. No.: 11/287,152

(22) Filed: Nov. 25, 2005

Related U.S. Application Data
(63) Continuation of application No. PCT/US04/17472, filed on Jun. 3, 2004.
(60) Provisional application No. 60/475,684, filed on Jun. 4, 2003.

(51) Int. Cl. A61K 38/18

(52) U.S. Cl. 514/12

(57) ABSTRACT

The present invention provides a method of treating or preventing retinopathy in an individual in need thereof, comprising administering to said individual an effective amount of an agonist of VEGFR-1 (vascular endothelial growth factor receptor-1). Another aspect of the invention provides a method of treating or preventing disorders associated with blood vessel loss, such as diabetic neuropathy, in an individual in need thereof, comprising administering to said individual an effective amount of an agonist of VEGFR-1. Preferred agonists of VEGFR-1 or analogs thereof are selected from the group consisting of PIGF-1 (placental growth factor-1), PIGF-2, VEGF-A, and VEGF-B. In one embodiment of the present invention, an agonist of VEGFR-1 is administered to prevent ROP in an individual in need thereof. In a preferred embodiment, the agonist is PIGF-1 or an analog thereof.

### CROSS REFERENCE

This application claims the benefit of U.S. Provisional Application No. 60/475,684, filed Jun. 04, 2003.

### GOVERNMENT SUPPORT

This invention was supported by National Institutes of Health grant EYO8670 and the government of the United States has certain rights thereto.

### FIELD OF THE INVENTION

The present application is directed to methods of treating and preventing retinopathies and disorders associated with blood vessel loss.

### BACKGROUND OF THE INVENTION

Retinopathy of prematurity (ROP) is a potentially blinding disease, initiated by lack of retinal vascular growth after premature birth. The greatest risk factor for development of ROP is low birth weight and gestational age but duration of oxygen exposure and oxygen levels also are contributing factors. ROP occurs in two phases (Simons, B. D. & Flynn, J. T. (1999) International Ophthalmology Clinics 39, 29-48). When infants are born prematurely the retina is incompletely vascularized. In infants who develop ROP, growth of vessels slows or ceases at birth leaving maturing but avascular and therefore hypoxic peripheral retina (Ashton, N. (1966) Am J Ophthalmol 62, 412-35; Flynn, J. T., O'Grady, G. E., Herrera, J., Kushner, B. J., Cantolino, S. & Milam, W. (1977) Arch Ophthalmol 95, 217-23). This is the first phase of ROP.

The extent of non-perfusion of the retina in the initial phase of ROP appears to determine the subsequent degree of neovascularization, the late destructive stage of ROP, with the attendant risk of retinal detachment and blindness (Penn, J. S., Tolman, B. L. & Henry, M. M. (1994) Invest Ophthalmol Vis Sci 35, 3429-35). If it were possible to allow blood vessels to grow normally in all premature infants, as they do in utero, the second damaging neovascular phase of ROP would not occur. When ROP was first described in 1942, the etiology was unknown. However, the liberal use of high supplemental oxygen in premature infants was soon associated with the disease and hyperoxia was shown to induce ROP-like retinopathy in neonatal animals with incompletely vascularized retinas. This suggested that an oxygen-regulated factor was involved. Expression of vascular endothelial growth factor (VEGF), which is necessary for normal vascular development, is oxygen-regulated and was found to be important for both phases of ROP (Aiello, L. P., Pierce, E. A., Foley, E. D., Takagi, H., Chen, H., Riddle, L., Ferrara, N., King, G. L. & Smith, L. E. (1995) Proc Natl Acad Sci USA 92, 10457-61; Robinson, G. S., Pierce, E. A., Rook, S. L., Foley, E., Webb, R. & Smith, L. E. (1996) Proc Natl Acad Sci USA 93, 4851-6; Pierce, E. A., Foley, E. D. & Smith, L. E. (1996) Arch Ophthalmol 114, 1219-28; Stone, J., Itin, A., Alon, T., Pe'er, J., Gnessin, H., Chan-Ling, T. & Keshet, E. (1995) J Neurosci 15, 4738-47; Alon, T., Hemo, I., Itin, A., Pe'er, J., Stone, J. & Keshet, E. (1995) Nature Medicine 1, 1024-8; Ozaki, H., Seo, M. S., Ozaki, K., Yamada, H., Yamada, E., Okamoto, N., Hofmann, F., Wood, J. M. & Campochiaro, P. A. (2000) American Journal of Pathology 156, 697-707). High supplemental oxygen affects the first phase of vascular growth in ROP animal models through suppression of VEGF expression. However, with careful use of moderate oxygen supplementation, the oxygen level in patients is a less significant risk factor for ROP than low birth weight, suggesting that other factors are also involved. (Kinsey, V. E., Arnold, H. J., Kalina, R. E., Stern, L., Stahlman, M., Odell, G., Driscoll, J. M., Jr., Elliott, J. H., Payne, J. & Patz, A. (1977) Pediatrics 60, 655-68; Lucey, J. F. & Dangman, B. (1984) Pediatrics 73, 82-96).

A complication of diabetes, diabetic retinopathy is a leading cause of blindness that affects approximately 25% of the estimated 16 million Americans with diabetes. It is believed that diabetic retinopathy is induced by hypoxia in the retina as a result of loss of blood vessels associated with hyperglycemia.

The degree of diabetic retinopathy is highly correlated with the duration of diabetes. There are two kinds of diabetic retinopathy. The first, non-proliferative retinopathy, is the earlier stage of the disease characterized by increased capillary dropout and increased permeability, microaneurysms, hemorrhages, exudates, and edema. Most visual loss during this stage is due to the fluid accumulating in the macula, the central area of the retina. This accumulation of fluid is called macular edema, and can cause temporary or permanent decreased vision. The second category of diabetic retinopathy is called proliferative retinopathy and is characterized by abnormal new vessel formation, which grows on the vitreous surface or extends into the vitreous cavity. Neovascularization can be very damaging because it can cause bleeding in the eye, retinal scar tissue, diabetic retinal detachments, or glaucoma, any of which can cause decreased vision or blindness.

Current treatment of non-proliferative retinopathy includes intensive insulin therapy to achieve normal glycemic levels in order to delay further progression of the disease, whereas the current treatment of proliferative retinopathy involves panretinal photocoagulation and vitrectomy. The treatment of non-proliferative retinopathy, while valid in theory, is mostly ineffective in practice because it usually requires considerable modification in the lifestyle of the patients, and many patients find it very difficult to maintain the near-normal glycemic levels for a time sufficient to slow and reverse the progression of the disease. Thus, the current treatment of non-proliferative retinopathy only delays the progression of the disease and cannot be applied effectively to all patients who require it.

The term “diabetic neuropathy” indicates a neuropathy associated with a chronic hyperglycemic condition. Diabetic neuropathy is roughly classified into groups of multiple neuropathy, autonomic neuropathy and single neuropathy. Diabetic neurosis usually indicates a symmetrical, distal, multiple neuropathy mainly causing sensory disturbance. Both multiple neuropathy and autonomic neuropathy are neuropathies characteristic of diabetics.

A cause for the diabetic neuropathies is a chronic hyperglycemic state. However, the mechanism of the crisis has not been fully elucidated yet. For the crisis mechanism of the neuropathy caused by hyperglycemia, there are two main theories, i.e. vascular dysfunction and disturbed metabolism.

According to the vascular dysfunction theory, the blood flow is disturbed by changes of the blood abnormalities (such as acceleration of platelet aggregation, increase of the blood viscosity and decrease of the red blood-cell deformity) or by changes of the blood vessel abnormalities (such as reduction of the production of nitric oxide from the endothelial cells of blood vessels and acceleration of the reactivity on vasoconstrictive substances), then the hypoxia of nerves is caused, and finally the nerves are degenerated. For example, when the platelet aggregation is accelerated by the chronic hyperglycemic state, the microvascular disturbance is caused to result in diabetic neuropathy.

It is commonly accepted that both the vascular dysfunction theory and the disturbed metabolism theory are correct. Also, it is considered that the disturbed metabolism mainly causes the initial stage of the diabetic neuropathy and, as the disease reaches an advanced stage, the concern of the vascular dysfunction increases. It has been shown that vascular endothelial growth factor (VEGF) can improve ischemic peripheral neuropathy both because of improvement of vessel function and improvement of nerve function itself (Schratzberger et al. Nature Medicine 6(4): 405-413, 2000).

Angiogenesis is characterized by excessive activity of VEGF. VEGF is actually comprised of a family of ligands (Klagsburn and D'Amore, Cytokine & Growth Factor Reviews 7:259-270, 1996). VEGF binds the high affinity membrane-spanning tyrosine kinase receptor KDR and the related fms-like tyrosine kinase-1, also known as Flt-1 or vascular endothelial cell growth factor receptor 1 (VEGFR-1). Cell culture and gene knockout experiments indicate that each receptor contributes to different aspects of angiogenesis. KDR mediates the mitogenic function of VEGF whereas VEGFR-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Improved methods of regulating neovascularization would be highly beneficial in treating disorders where blood vessel loss is a problem as well as retinopathies, since blood vessel loss precedes proliferative retinopathy.

### SUMMARY OF THE INVENTION

We show here the surprising result that specific activation of VEGFR-1 protects against oxygen-induced vessel loss without stimulating vascular proliferation and neovascularization in vivo.

The present invention provides a method of treating or preventing retinopathy in an individual in need thereof, comprising administering to said individual an effective amount of an agonist of VEGFR-1 (vascular endothelial growth factor receptor-1). Preferably the agonist is specific to VEGFR-1.

Another aspect of the invention provides a method of treating or preventing disorders associated with blood vessel loss, such as diabetic neuropathy, in an individual in need thereof, comprising administering to said individual an effective amount of an agonist of VEGFR-1. Preferably the agonist is specific to VEGFR-1.

Preferred specific agonists of VEGFR-1 include, for example, PIGF-1 (placental growth factor-1), and analogs and peptide mimetics thereof. Non-specific agonists may be used as long as they are used at concentrations that preferentially activate VEGFR-1. Preferred non-specific agonists include, for example, PIGF-2, VEGF-A, VEGF-B, and analogs and peptide mimetics thereof.

In one embodiment of the present invention, an agonist of VEGFR-1 is administered to prevent ROP in an individual in need thereof. In a preferred embodiment, the agonist is PIGF-1 or an analog thereof.

In yet another aspect of the invention there is provided use of an agonist of VEGFR-1 in the manufacture of a medicament for treating and/or preventing retinopathy and/or disorders associated with blood vessel loss.

Finally, there is also provided an article of manufacture comprising packaging material and a pharmaceutical agent contained within the packaging material. The packaging material comprises a label which indicates that the pharmaceutical may be administered, for a sufficient term at an effective dose, for treating and/or preventing retinopathy and/or disorders associated with blood vessel loss. The pharmaceutical agent comprises an agonist of VEGFR-1 together with a pharmaceutically acceptable carrier.

It is to be understood that while the invention has been described in conjunction with the preferred specific embodiments thereof that the foregoing description as well as the examples that follow are intended to illustrate and not limit the scope of the invention. Other aspects, advantages and modifications within the scope of the invention will be apparent to those skilled in the art to which the invention pertains.

### BRIEF DESCRIPTION OF THE DRAWINGS

FIGS. 1a and 1b show real-time RT-PCR quantification of VEGFR-1 and VEGFR-2 mRNA during retinal vascular development. Copy numbers of VEGFR-1 and VEGFR-2 mRNA/10⁶ cyclophylin control from total retinal RNA at specific time points were measured. (FIG. 1a) VEGFR-1 mRNA expression increases linearly with retinal vascular development and the expression increases 60-fold at P26 versus P3. (FIG. 1b) VEGFR-2 mRNA expression decreases modestly (<15%) during retinal vessel development. The ratio of VEGFR-2 RNA to VEGFR-1 mRNA expression ranges from 200-fold at P3 to 2-fold at P26.

FIGS. 2a-2b show that PIGF-1, but not VEGF-E, prevents hyperoxia-induced retinal vessel loss, thus implicating VEGFR-1 in survival (FIG. 2a). P8 FITC-dextran perfused retinal flat mount retina from room air (normoxia) treated control mice (normoxia) or hyperoxia-treated mice (75% O₂ for 17 hours P7-P8) after intravitreal injections at P7 of (hyperoxia) control BSS (P7) in one eye and (hyperoxia+PIGF-1) VEGFR-1 specific ligand PIGF-1 in the contralateral eye. Vessels delineated with FITC show that PIGF-1 confers significant protection from oxygen-induced vessel loss compared to BBS control. Analysis of non-vascularized area shows greater than a 4-fold difference between mice treated with PIGF-1 (22.2±3.4% vascularized area) and mice treated with BSS (5.1±1.2%) (n=6, P<0.001).

(FIG. 2b) FITC-dextran perfused retinal flat mount at P8 control retina from room air-treated (normoxia) mice or oxygen-exposed mice (75% O₂ for 17 hours P7-P8) after intravitreal injections at P7 of (hyperoxia) control BSS in one eye and (hyperoxia+VEFG-E) VEGFR-2 specific ligand VEGF-E in the contralateral eye. Analysis of non-vascularized area shows no significant difference between VEGF-E and BSS treated eyes (n=6, p=0.87). Results are representative of two independent experiments.

FIGS. 3a-3c shows that activation of VEGFR-1 by PIGF-1 does not increase normal retinal vessel growth or revascularization. (FIG. 3a) Intravitreal injections at P3 with control BSS in one eye and PIGF-1 in the contralateral eye (n=6). Retinal vessel growth area was measured in whole-mount at P5. PIGF-1 injected eyes had a mean of 41.67±5.63% of the retina vascularized. Similarly, the BSS injected contralateral control eyes were vascularized 42.18±8.60% (p=0.91). Results are representative of two independent experiments. (FIG. 3b) Vessel revascularization was measured at P15 mice after induction of vessel loss by oxygen (P7-P12) followed by intravitreal injections of control saline (BSS) at P13 in one eye and PIGF-1 in the contralateral eye. PIGF-1 injected eyes were 26.32±2.62% vascularized; and, similarly, BSS-treated contralateral control eyes were 26.29±2.86% vascularized (n=6, p=0.99). Results are representative of two independent experiments. (FIG. 3c) In eyes with oxygen-induced retinopathy, the mean number of vascular nuclei extending into the vitreous at P17 in 10 retinal cross-sections per eye (N=8 eyes) was counted after intravitreal injections of control saline BSS at P13 (after 5 days P7-P12 of 75% O₂ treatment) in one eye and PIGF-1 in the contralateral eye. Both BSS and PIGF-1-injected eyes showed means of 9.98 and 9.96 vascular nuclei (p=0.61), respectively, indicating no stimulation of proliferation by PIGF-1.

### DETAILED DESCRIPTION OF THE INVENTION

“PIGF-1, PIGF-2, VEGF-A, and VEGF-B” refers to growth factors from any species, including bovine, ovine, porcine, equine, and human, preferably human, and, if referring to exogenous administration, from any source, whether natural, synthetic, or recombinant, provided that it will bind VEGFR-1.

A “therapeutic composition,” as used herein, is defined as comprising an agonist of VEGFR-1 or an analog thereof. The therapeutic composition may also contain other substances such as water, minerals, carriers such as proteins, and other excipients known to one skilled in the art.

“Analogs” are compounds having the same therapeutic effect in humans or animals. These can be naturally occurring analogs (e.g., truncated) or any synthetic analogs.

“Agonists” are growth factors and compounds, including peptides, small molecules, and structural or functional mimetics, which are capable of activating VEGFR-1.

Biologically active derivatives or analogs of the agonists described herein also include peptide mimetics. Peptide mimetics can be designed and produced by techniques known to those of skill in the art. (see e.g., U.S. Pat. Nos. 4,612,132; 5,643,873 and 5,654,276, the teachings of which are incorporated herein by reference). These mimetics can be based, for example, on the protein's specific amino acid sequence and maintain the relative position in space of the corresponding amino acid sequence (Iyer, S. et al., (2001) Journal of Biological Chemistry 276 (15): 12153-12161). These peptide mimetics possess biological activity similar to the biological activity of the corresponding peptide compound, but possess a “biological advantage” over the corresponding amino acid sequence with respect to one, or more, of the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Modifications of peptides to produce peptide mimetics are described in U.S. Pat. Nos. 5,643,873 and 5,654,276, the teachings of which are incorporated herein by reference. Other forms of the proteins or polypeptides described herein and encompassed by the present invention, include those which are “functionally equivalent.” This term, as used herein, refers to any nucleic acid sequence and its encoded amino acid which mimics the biological activity of the protein or polypeptide and/or functional domains thereof.

The invention provides screening assay methods for identifying therapeutic compounds useful for treatments which activate VEGFR-1 and for the treatment and/or prevention of retinopathies and disorders associated with blood vessel loss in human patients. These methods include measuring the effect of a potential therapeutic compound on the prevention of oxygen induced vessel loss or monocyte migration. The screening assay methods of the invention simplify the evaluation, identification and development of candidate compounds and therapeutic agents for the treatment of such conditions and disorders. In general, the screening methods provide a simplified means for selecting natural product extracts or compounds of interest from a large population, generally a compound library, which are further evaluated and condensed to a few active and selective materials useful for treatments of such conditions and disorders (these treatments are sometimes referred to herein as the “desired purposes of the invention”).

Constituents of this pool are then purified, evaluated, or modified by combinatorial chemistry in order to identify preferred compounds for the desired purposes of the invention.

Compounds that modulate the biological activity of VEGFR-1 can be identified by their effects on a known biological activity of the receptor.

We have now discovered a method of treating and/or preventing retinopathies and disorders associated with blood vessel loss, such as diabetic neuropathy, in an individual in need of such treatment. This method involves administering to the individual in need an effective amount of an agonist of VEGFR-1. In a preferred embodiment, the agonist is a growth factor or analog thereof selected from the group consisting of PIGF-1, PIGF-2, VEGF-A, and VEGF-B. Most preferably the agonist is PIGF-1.

The inventive methods disclosed herein provide for the parenteral and oral administration of an agonist of VEGFR-1 to an individual in need of such treatment. Parenteral administration includes, but is not limited to, intravenous (IV), intramuscular (IM), subcutaneous (SC), intraperitoneal (IP), intranasal, and inhalant routes. In the method of the present invention an agonist of VEGFR-1 or an analog thereof is preferably administered orally. IV, IM, SC, and IP administration may be by bolus or infusion, and may also be by slow release implantable device, including, but not limited to pumps, slow release formulations, and mechanical devices. The formulation, route and method of administration, and dosage will depend on the disorder to be treated and the medical history of the patient. In general, a dose that is administered by subcutaneous injection will be greater than the therapeutically-equivalent dose given intravenously or intramuscularly.

For parenteral or oral administration, compositions may be semi-solid or liquid preparations, such as liquids, suspensions, and the like. Physiologically compatible carriers are those that are non-toxic to recipients at the dosages and concentrations employed and are compatible with other ingredients of the formulation. For example, the formulation preferably does not include oxidizing agents and other compounds that are known to be deleterious to polypeptides. Hence, physiologically compatible carriers include, but are not limited to, normal saline, serum albumin, 5% dextrose, plasma preparations, and other protein-containing solutions. Optionally, the carrier may also include detergents or surfactants.

In yet another aspect of the invention there is provided use of an agonist of VEGFR-1 in the manufacture of a therapeutic composition for treating or preventing retinopathy or a disorder associated with blood vessel loss.

Finally, there is also provided an article of manufacture comprising packaging material and a pharmaceutical agent contained within the packaging material. The packaging material comprises a label which indicates that the pharmaceutical may be administered, for a sufficient term at an effective dose, for treating and/or preventing retinopathy or a disorder associated with blood vessel loss. The pharmaceutical agent comprises an agonist of VEGFR-1 together with a pharmaceutically acceptable carrier.

For therapeutic applications, an agonist of VEGFR-1 may be suitably administered to a patient, alone or as part of a pharmaceutical composition, comprising the agonist of VEGFR-1 together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for oral, nasal, topical (including buccal and sublingual), or parenteral (including subcutaneous, intramuscular, intravenous and intradermal) administration. The formulations may conveniently be presented in unit dosage form, e.g., tablets and sustained release capsules, and in liposomes, and may be prepared by any methods well know in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

The invention will be further characterized by the following examples which are intended to be exemplary of the invention.

### EXAMPLE

Methods

FITC-dextran perfusion and retinal whole-mount. C57B1/6 mice were anesthetized with Avertin (Sigma, St. Louis, Mo.) and sacrificed by intracardiac perfusion with 4% paraformaldehyde and 2×10⁶ mol wt fluorescein (FITC)-dextran in PBS (10). Eyes were enucleated and fixed in 4% paraformaldehyde for 2 h at 4° C. The retinas were isolated and either directly whole-mounted with glycerol-gelatin (Sigma) onto poly-lysin coated slides with the photoreceptor side up, or mounted after in-situ hybridization or immunohistochemical staining. The retinas were examined with a fluorescence microscope (Olympus, Tokyo, Japan). Images were digitized using a 3 CCD color video camera (DX-950P, Sony) and processed with Northern Eclipse software (Toronto, Canada).

O₂-induced vessel degeneration. To induce vessel loss, postnatal day 7 (P7) C57B1/6 mice with their nursing mother were exposed to 75% oxygen for times ranging from 17 h to 5 days (11). At P7, human PIGF-1 (0.5 μg/0.5 μl BSS) (R&D Systems, Minneapolis, Minn.) or VEGF-E (0.5 μg/0.5 μl BSS) (Cell Science, Norwood, Mass.) was intravitreally injected into one eye and control 0.5 μl BSS (balanced salt solution) in the contralateral eye (n=6). After 17 h of O₂ exposure, eyes were collected after FITC-dextran perfusion and retinas were whole-mounted onto slides as described.

Whole-mount in situ hybridization. Sense (control) and anti-sense mRNA probes for VEGF-A, VEGFR-1 and VEGFR-2 were transcribed in vitro by using digoxigenin-UTP labeling kit according to the manufacturer's protocol (Roche, Indianapolis, Ind.). Retinas were pre-incubated with 0.2 M HCl to remove endogenous alkaline phosphatase activity, digested with proteinase K (20 μg/ml) in PBS buffer, post-fixed in 4% paraformaldehyde-PBS, and treated with 0.1 M triethanolamine containing 0.25% acetic anhydride. Retinas were prehybridized in 50% formamide containing dextran sulfate, ssDNA, and tRNA in phosphate buffer, pH 7.5 for 1 h at 50° C. and then hybridized with 100 ng/ml Digoxygenin-labeled RNA probe at 50° C. overnight. Antibody to Digoxygenin (1:1000) (Roche) was applied for 4 h at room temperature and color developed with alkaline phosphatase substrate (Roche) for 10 min at room temperature. Retinas were flat-mounted as described.

Immunohistochemical staining. Eyes were fresh frozen in OCT (Fisher Scientific, Pittsburgh, Pa.), cut into 14-μm sections, fixed with methanol, rinsed with PBS, then blocked with PBS/0.5% Triton/1% bovine serum albumin. The sections were stained with primary antibodies against VEGFR-1 or VEGFR-2 (R&D Systems), and biotin-labeled endothelial cell specific isolectin, griffonia simplicifolia I (Vector, Burlingame, Calif.) for 2 h at room temperature. The secondary reagents used were anti-goat-cy3, anti-goat-FITC (Sigma), avidin-AMCA or avidin-Texas red (Vector). For whole-mount immunohistochemical staining, 4% paraformaldehyde-fixed (2 hours) retinas were rinsed in PBS, then blocked and stained according to procedures described for cross-section. Images were captured with a 3 CCD color Sony video camera (DX-950P) and processed with Northern Eclipse software.

RNA Isolation and cDNA Preparation. For each of the following developmental time points: P3, P5, P7, P8, P12, P15, P17, P26 and P33, total RNA was extracted by RNAeasy kit (Qiagen, Chatsworth Calif.) from the retinas of one mouse from each of 12 litters and then pooled to reduce biologic variability. Retinas from each time point were lysed in guanadinium isothiocyanate lysis buffer following manufacturer's instruction and RNA was suspended in DEPC treated H₂O. To generate cDNA, 1 μg total RNA was treated with DNase I (Ambion, Austin, Tex.) to remove any contaminating genomic DNA. The DNase-treated RNA (100 ng) was then converted into cDNA by using murine leukemia virus reverse transcriptase (Gibco BRL Life Technologies). All cDNA samples were aliquoted and stored at −80° C.

Quantitative Real-time RT-PCR analysis of gene expression. Real-time PCR primers (Genemed Synthesis, South San Francisco, Calif.) targeting murine VEGFR-1, VEGFR-2, and cyclophilin were designed using Primer Express software (Applied BioSystems, Foster City, Calif.). Cyclophilin expression was unchanged during retinal development and was used as the reference standard (normalizer). Specificity of each primer was determined with NCBI Blast module. Efficacy of each primer set was assured by testing amplicons for the specific melting point temperatures (Primer Express, Applied BioSystems Software). The ABI Prism 7700 Sequence Detection System (Applied Biosystems) and the SYBR Green master mix kit (Applied Biosystems) were used for detecting real-time PCR products from 0.25-2.5 ng reverse transcribed cDNA samples (12). To determine absolute copy numbers of murine VEGFR-1 and VEGFR-2 mRNA, we cloned and isolated individual cDNA templates of VEGFR-1, VEGFR-2 and cyclophilin that cover the sequences bracketed by the real-time PCR primers and determined absolute mRNA numbers as described (12, 13). Standard curves for each gene were plotted with quantified cDNA template during each real-time PCR reaction. Each target gene mRNA copy number was then normalized to 10⁶ copies of cyclophilin control.

The sequences of the PCR primer pairs (5′ to 3′) that were used for each gene are as follows:

VEGFR-1:

(SEQ ID NO: 1)

forward, 5′- GAGGAGGATGAGGGTGTCTATAGGT -3′

(SEQ ID NO: 2)

reverse, 5′- GTGATCAGCTCCAGGTTTGACTT -3′
VEGFR-2:

(SEQ ID NO: 3)

forward, 5′- GCCCTGCCTGTGGTCTCACTAC -3′

(SEQ ID NO: 4)

reverse, 5′- CAAAGCATTGCCCATTCGAT -3′
Cyclophilin:

(SEQ ID NO: 5)

forward, 5′- CAGACGCCACTGTCGCTTT -3′

(SEQ ID NO: 6)

reverse, 5′- TGTCTTTGGAACTTTGTCTGCAA -3′;

Analysis of retinal vessel growth and retinal revascularization. Early retinal vessel development. At P3, PIGF-1 (0.5 μg/0.5 μl BSS) was injected into the vitreous of one eye and control, 0.5 μl BSS, injected into the contralateral eye (n=6). 48 hours later (P5) retinas were harvested after FITC-dextran perfusion (10), whole-mounted, images captured by Sony (DX-950P) color video camera and vessel growth measured by Northern Eclipse software and expressed as a percentage of total retinal area (14). To show that the system could detect an increase in vascular growth with cytokine stimulation we examined a positive control (12) consisting of cells expressing high levels of VEGF-A injected into one eye and saline into the contralateral eyes of 6 mice at a) P7 followed by flat mounting of retina at P12. b) injection at P12 followed by flat mounts at P15 c) injection at P15 followed by retinal flat mounts at P17. VEGF-A significantly increased vessel density with exposure from P7-P12 showing that the method could detect a positive change (data not shown).

Retinal revascularization after oxygen-induced vessel loss: P7 mice were exposed to 75% O₂ for 5 days to induce vessel loss and returned to room air at P12 (11). After 24 hours (P13), mice were injected intravitreally with PIGF-1 (0.5 μg/0.5 μl BSS) in one eye versus 0.5 μl BSS in the contralateral eye (n=6). Retinas were isolated at P15 after FITC-dextran perfusion, whole-mounted, and vascularized area measured as above to determine the effect of PIGF-1 on revascularization. b) Positive control retinal flat mounts showed a significant increase in vessel density at P15 with VEGF A treatment (data not shown)

Retinal neovascularization after oxygen-induced vessel loss: P13 mice, after 5-day oxygen exposure from P7 to P12 (11), were injected intravitreally with PIGF-1 (0.5 μg/0.5 μl BSS) in one eye and control 0.5 μl BSS in the contralateral eye (n=8). Retinas were harvested at P17 and the mean number of neovascular nuclei extending into the vitreous were counted in retinal cross-sections as previously described (5, 11, 14). c) Positive control retinal flat mounts showed a significant increase in vessel growth at P17 with VEGF A treatment (data not shown)

Results

VEGFR-1, VEGFR-2, and VEGF-A mRNA expression during retinal vessel development. Early in retinal vascular development, at five days post-partum (P5), whole-mount in-situ hybridization identified VEGF-A mRNA expression in areas of physiological hypoxia anterior to the growing vessel front and suppression of VEGF-A expression was found posterior to the growing vessel front which coincided with more mature retinal vessels, whereas VEGFR-2 mRNA expression was uniform over the entire retina. Hybridization with control sense RNAs for VEGF-A, VEGFR-2 and VEGFR-1 showed no significant background. At P7, when superficial retinal vessels have extended further into the periphery, VEGF-A mRNA was observed anterior to vessels in the same pattern as that observed at P5. Similarly, the pattern seen at P5 with VEGFR-1 and VEGFR-2 was also seen at P7.

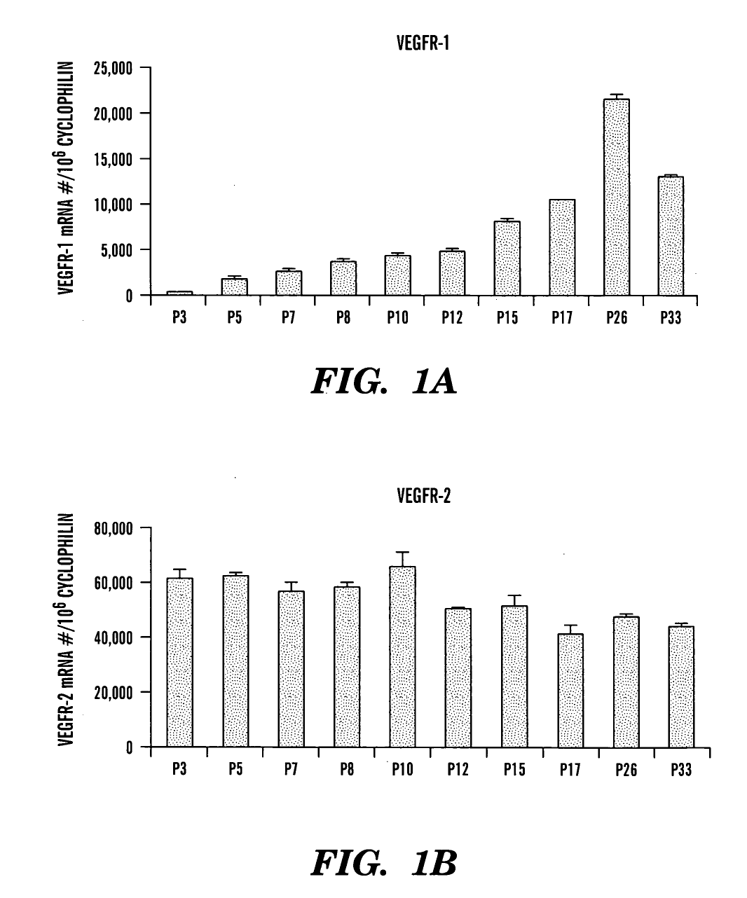

VEGFR-1 and VEGFR-2 mRNA expression from whole retina during vessel development was examined by quantitative real-time RT-PCR. VEGFR-1 mRNA expression increased linearly during the course of retina vessel development (FIG. 1a). At P3, when retinas are still largely avascular, 350 copies of VEGFR-1 mRNA/10⁶ copies of cyclophilin (internal control) were detected. By P26, when retinal vessels are nearly fully developed, VEGFR-1 mRNA expression had increased 60-fold (22,000 copies VEGFR-1 mRNA/10⁶ copies of cyclophilin). Expression declined at P33 when retinal vessels were fully developed, falling to 13,000 VEGFR-1 mRNA copies/10⁶ copies of cyclophilin. This result provides further evidence of a relationship between VEGFR-1 expression and retinal vessel formation. Quantification of VEGFR-2 mRNA revealed sharp contrasts with VEGFR-1 mRNA expression. Early in retinal vascular development (P3), VEGFR-2 mRNA expression was about 180-fold higher than VEGFR-1 (60,000 VEGFR-2 versus 350 VEGFR-1 mRNA copies/10⁶ copies of cyclophilin). Moreover, this ratio declined with retinal vascular development and reached a ratio of ˜2:1 when retina are fully vascularized at P26 (45,000 versus 22,000 VEGFR-2 mRNA copies/10⁶ copies of cyclophilin) (FIG. 1b).

Immunohistochemical localization of VEGFR-1 and VEGFR-2 proteins in developing retina. In retinal whole-mounts of P5 mice, VEGFR-1, VEGFR-2, and endothelial cells were visualized with immunohistochemical staining with VEGFR-1 antibody, VEGFR-2 antibody and griffonia simplicifolia I isolectin, respectively. VEGFR-1 antibody staining coincided with retinal vessels at P5, whereas VEGFR-2 antibody primarily stained non-vascular cells of the neural retina. In merged images, the VEGFR-1 positive cells completely overlapped with isolectin positive retinal vessels. In contrast, VEGFR-2 positive cells appeared in the interstices between isolectin positive retinal vessels, indicating that VEGFR-2 expression is primarily associated with the neural retina.

To confirm these findings, we examined at P5 VEGFR-1 and VEGFR-2 protein localization in retinal cross section. VEGFR-1 antibody prominently stained cells in the ganglion cell layer. Staining coincided with vascular endothelial cells identified with endothelial cell specific isolectin. VEGFR-2 antibody also stained cells prominently in the ganglion cell layer and the inner/outer nuclear cell layers of P5 retinal cross-sections. However, VEGFR-2 antibody staining did not co-localize with vascular endothelial cells but instead localized to neural retina, thus verifying our findings in whole mounts. This neural-cell-specific expression of VEGFR-2 was limited to perinatal mice. By P12, when blood vessels are more fully developed into 3 layers, vascular endothelial cell staining began to coincide with some but not the majority of anti-VEGFR-2 staining (as well as with VEGFR-1). VEGFR-2 was still found in neural retina. By P15, vascular staining with VEGFR-2 became more evident, as VEGFR-1 continued to be specifically expressed on endothelial cells. VEGFR-2 can be seen on cells which span the thickness of the retina consistent with Muller cell morphology. Overall, these results indicate that in neonatal retina VEGFR-1 expression is specific to retinal blood vessels, whereas VEGFR-2 is predominantly expressed in neural cells and developmentally regulated in blood vessels.

VEGFR-1 specific agonist PIGF-1 protects against oxygen-induced vessel loss; VEGFR-2 specific agonist VEGF-E is not protective. PIGF-2 is the only PIGF isoform produced in the mouse, is a ligand for VEGFR-1 but it is also a ligand for neuropilin (15), a VEGF receptor associated with angiogenesis (16). In order to define specific functions of VEGFR-1 and VEGFR-2 in hyperoxia-induced retinal vessel loss, we administered the VEGFR-1 specific ligand, PIGF-1 (6, 7) and the VEGFR-2 specific ligand, VEGF-E (8, 9) by intravitreal injection. PIGF-1 does not bind to neuropilin unlike PIGF-2. VEGF-E binds only VEGFR-2 and does not bind neuropilin. Thus with the use of these specific ligands we could eliminate confounding effects of neuropilin binding.

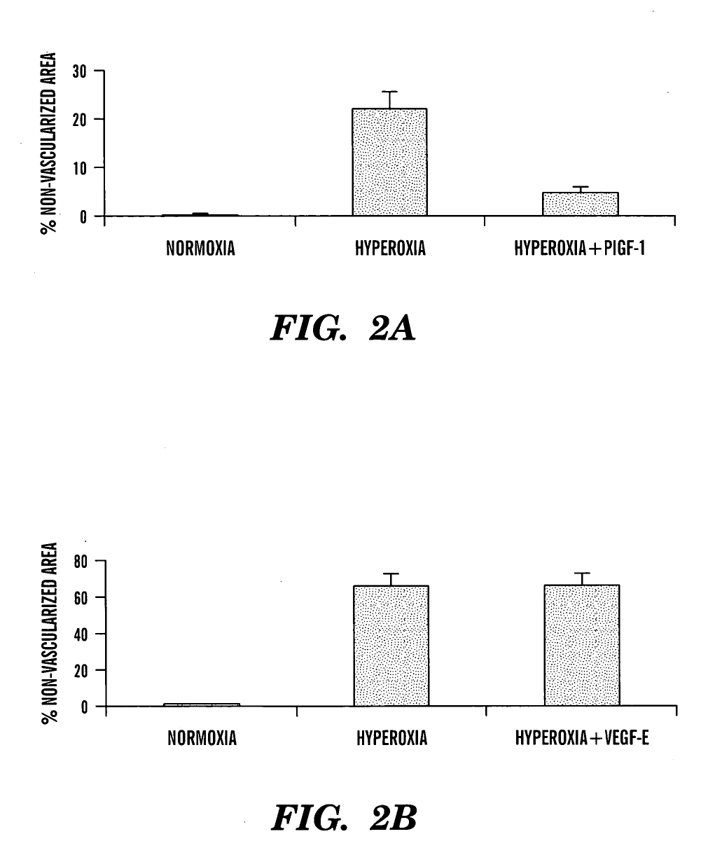

Analysis of vascularized/non-vascularized area indicated a ˜77% reduction in hyperoxia-induced vessel loss associated with PIGF-1 intravitreal injections in comparison with controls (FIG. 2a). Specifically, 17 hour hyperoxia-treated eyes had 22.2±3.4% non-vascularized area, whereas contralateral PIGF-1-injected eyes showed only 5.1±1.2% non-vascularized area (P<0.001 8). In contrast, VEGF-E provided no detectable protective effect against O₂-induced vessel loss compared to control BSS-injected contralateral eyes (FIG. 2b). Thus, these experiments demonstrate that PIGF-1, which specifically binds VEGFR-1 protects postnatal mice retina from hyperoxia-induced vaso-obliteration whereas activation of VEGFR-2 by VEGF-E is not protective.

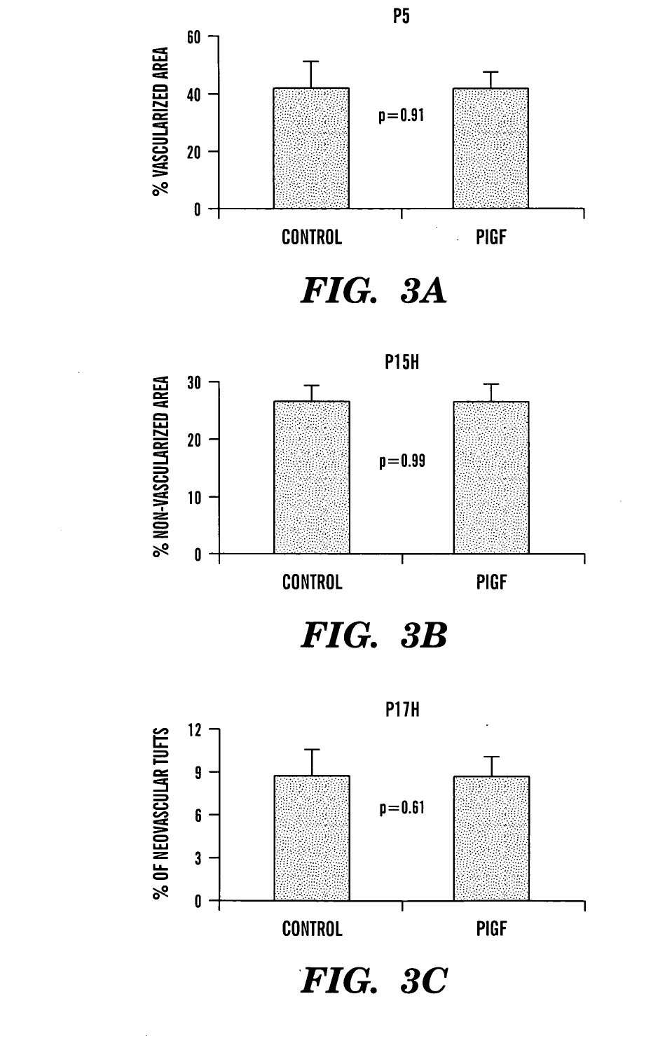

PIGF-1 does not stimulate murine retinal vessel growth. Because PIGF-1 protected against hyperoxia-induced retinal vessel loss, we explored whether PIGF-1 at similar concentration also stimulated retinal vessel growth in vivo under conditions of normal vessel development and during re-growth of vessels after O₂-induced vessel loss at P15. Intravitreal administration of PIGF-1 (0.5 μg/0.5 μl BSS) at P3 resulted in no significant stimulation of normal vessel growth compared to control eyes when examined 2 days later at P5 (n=6, P=0.91) (FIG. 3a). In addition, administration of PIGF-1 at P13, after 5-days of 75% O₂ exposure from P7 to P12, did not stimulate subsequent re-growth of vessels as measured at P15 (P=0.99) (FIG. 3b). Similarly, intravitreal injections of PIGF-1 at P13 also did not affect retinal neovascularization measured at P17 (FIG. 3c). Thus, these findings indicate that PIGF-1 at concentrations which prevent vessel loss does not stimulate retinal vascular proliferation and neovascularization in vivo.

Discussion

Although VEGF-A is known to promote survival of retinal vessels under hyperoxia, the role of specific VEGF-A receptors in this process has not been described. Through utilization of receptor-specific ligands, this study has distinguished the contributions of VEGF-A receptors VEGFR-1 and VEGFR-2 to the survival of retinal blood vessels in neonatal mice. Specifically, we find that PIGF-1, a VEGFR-1-specific agonist, is critical to vessel survival and does not promote vaso-proliferation. These findings suggest the important possibility of treating premature infants with VEGFR-1-specific ligands in order to prevent retinal ischemia without provoking vaso-proliferation.

Our study indicates that concentrations of PIGF-1, sufficient to prevent oxygen-induced vessel loss, did not increase retinal vessel proliferation during normal retinal vascular development, nor did such concentrations of PIGF-1 increase re-growth of vessels after oxygen-induced loss, nor increase hypoxia-induced vaso-proliferation. Thus, our findings identify selective functions for PIGF-1 and VEGFR-1 in supporting retinal blood vessel survival in neonates.

VEGFR-1 activation is not necessary for normal vascular development since deletion of the tyrosine kinase domain of VEGFR-1 allows normal embryonic angiogenesis (17). Deletion of the entire VEGFR-1 gene is embryonically lethal due to abnormal vascular development. These abnormalities may be attributable to loss of soluble VEGFR-1, which is a natural competitor for VEGF binding to VEGFR-2 (18). Although at low concentrations we found no increase in vaso-proliferation with PIGF-1, some studies suggest the possibility that, in other settings, VEGFR-1 may play a role in pathological vaso-proliferation (19-21). Our studies suggest that in some cases what appears to be VEGFR-1 induced increased proliferation may be due to increased vascular survival. In order to specifically define the role of VEGFR-1, we chose to target VEGFR-1 with PIGF-1 which binds VEGFR-1 and does not bind neuropilin unlike PIGF-2, which binds both receptors. Neuropilin is critically involved in vascular development (15, 16) and studies with PIGF-2 are more difficult to interpret because of neuropilin binding.

Consistent with the specific importance of VEGFR-1 for blood vessel survival in neonatal retina, we found that VEGFR-1 is localized to blood vessels but that VEGFR-2 is localized primarily to the neural retina. Our observation that VEGFR-2 is expressed primarily outside the vasculature in the neonatal retina is in accord with a growing body of evidence showing the importance of VEGF receptors in neural cells (14, 22-26). Interestingly, this selective localization of VEGFR-2 expression to the neural retina does not persist. By P12, VEGFR-2 is also seen on retinal vessels. This change of VEGFR-2 expression with retinal development suggests coordinated regulation of VEGF activity towards neural cells and vascular endothelial cells.

In summary, experiments described here identify a prominent association between VEGFR-1 and neonatal blood vessels in retina, and they establish that specific stimulation of VEGFR-1 with low doses of PIGF-1 protects retinal vessels from oxygen-induced degeneration without promoting vascular growth. In contrast to VEGFR-1, VEGFR-2 is predominantly expressed in neural retina, and specific stimulation of VEGFR-2 with VEGF-E does not prevent oxygen-induced vaso-obliteration. Thus, these studies define important distinctions between VEGFR-1 and VEGFR-2 in neonatal retina, and they identify specific stimulation of VEGFR-1 as an attractive strategy for preventing the early degenerative stage of ROP.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

All references described herein are incorporated herein by reference.

### REFERENCES

1. Gibson, D. L., Sheps, S. B., Uh, S. H., Schechter, M. T., and McCormick, A. Q. 1990. Retinopathy of prematurity-induced blindness: birth weight-specific survival and the new epidemic. Pediatrics 86:405-412.
2. Bossi, E., and Koerner, F. 1995. Retinopathy of prematurity. Intensive Care Med 21:241-246.
3. Alon, T., Hemo, I., Itin, A., Pe'er, J., Stone, J., and Keshet, E. 1995. Vascular endothelial growth factor acts as a survival factor for newly formed retinal vessels and has implications for retinopathy of prematurity. Nature Medicine 1: 1024-1028.
4. Pierce, E. A., Foley, E. D., and Smith, L. E. 1996. Regulation of vascular endothelial growth factor by oxygen in a model of retinopathy of prematurity [see comments] [published erratum appears in Arch Ophthalmol March 1997; 115(3):427]. Arch Ophthalmol 114:1219-1228.
5. Aiello, L. P., Pierce, E. A., Foley, E. D., Takagi, H., Chen, H., Riddle, L., Ferrara, N., King, G. L., and Smith, L. E. 1995. Suppression of retinal neovascularization in vivo by inhibition of vascular endothelial growth factor (VEGF) using soluble VEGF-receptor chimeric proteins. Proc Natl Acad Sci USA 92:10457-10461.
6. Park, J. E., Chen, H. H., Winer, J., Houck, K. A., and Ferrara, N. 1994. Placenta growth factor. Potentiation of vascular endothelial growth factor bioactivity, in vitro and in vivo, and high affinity binding to Flt-1 but not to Flk-1/KDR. J Biol Chem 269:25646-25654.
7. Persico, M. G., Vincenti, V., and DiPalma, T. 1999. Structure, expression and receptor-binding properties of placenta growth factor (PIGF). Curr Top Microbiol Immunol 237:31-40.
8. Meyer, M., Clauss, M., Lepple-Wienhues, A., Waltenberger, J., Augustin, H. G., Ziche, M., Lanz, C., Buttner, M., Rziha, H. J., and Dehio, C. 1999. A novel vascular endothelial growth factor encoded by Orf virus, VEGF-E, mediates angiogenesis via signalling through VEGFR-2 (KDR) but not VEGFR-1 (Flt-1) receptor tyrosine kinases. Embo J 18:363-374.
9. Ogawa, S., Oku, A., Sawano, A., Yamaguchi, S., Yazaki, Y., and Shibuya, M. 1998. A novel type of vascular endothelial growth factor, VEGF-E (NZ-7 VEGF), preferentially utilizes KDR/Flk-1 receptor and carries a potent mitotic activity without heparin-binding domain. J Biol Chem 273:31273-31282.
10. D'Amato, R., Wesolowski, E., and Smith, L. E. 1993. Microscopic visualization of the retina by angiography with high-molecular-weight fluorescein-labeled dextrans in the mouse. Microvasc Res 46:135-142.
11. Smith, L. E., Wesolowski, E., McLellan, A., Kostyk, S. K., D'Amato, R., Sullivan, R., and D'Amore, P. A. 1994. Oxygen-induced retinopathy in the mouse. Invest Ophthalmol Vis Sci 35:101-111.
12. Shih, S. C., Robinson, G. S., Perruzzi, C. A., Calvo, A., Desai, K., Green, J. E., Ali, I. U., Smith, L. E., and Senger, D. R. 2002. Molecular profiling of angiogenesis markers. Am J Pathol 161:35-41.
13. Calvo, A., Yokoyama, Y., Smith, L. E., Ali, I., Shih, S. C., Feldman, A. L., Libutti, S. K., Sundaram, R., and Green, J. E. 2002. Inhibition of the mammary carcinoma angiogenic switch in C3(1)/SV40 transgenic mice by a mutated form of human endostatin. Int J Cancer 101:224-234.
14. Robinson, G. S., Ju, M., Shih, S. C., Xu, X., McMahon, G., Caldwell, R. B., and Smith, L. E. 2001. Nonvascular role for VEGF: VEGFR-1, 2 activity is critical for neural retinal development. Faseb J 15:1215-1217.
15. Migdal, M., Huppertz, B., Tessler, S., Comforti, A., Shibuya, M., Reich, R., Baumann, H., and Neufeld, G. 1998. Neuropilin-1 is a placenta growth factor-2 receptor. J Biol Chem 273:22272-22278.
16. Takashima, S., Kitakaze, M., Asakura, M., Asanuma, H., Sanada, S., Tashiro, F., Niwa, H., Miyazaki Ji, J., Hirota, S., Kitamura, Y., et al. 2002. Targeting of both mouse neuropilin-1 and neuropilin-2 genes severely impairs developmental yolk sac and embryonic angiogenesis. Proc Natl Acad Sci USA 99:3657-3662.
17. Hiratsuka, S., Minowa, O., Kuno, J., Noda, T., and Shibuya, M. 1998. Flt-1 lacking the tyrosine kinase domain is sufficient for normal development and angiogenesis in mice. Proc Natl Acad Sci USA 95:9349-9354.
18. Kendall, R. L., and Thomas, K. A. 1993. Inhibition of vascular endothelial cell growth factor activity by an endogenously encoded soluble receptor. Proc Natl Acad Sci USA 90:10705-10709.
19. Carmeliet, P., Moons, L., Luttun, A., Vincenti, V., Compernolle, V., De Mol, M., Wu, Y., Bono, F., Devy, L., Beck, H., et al. 2001. Synergism between vascular endothelial growth factor and placental growth factor contributes to angiogenesis and plasma extravasation in pathological conditions. Nat Med 7:575-583.
20. Adini, A., Kornaga, T., Firoozbakht, F., and Benjamin, L. E. 2002. Placental growth factor is a survival factor for tumor endothelial cells and macrophages. Cancer Res 62:2749-2752.
21. Hiratsuka, S., Maru, Y., Okada, A., Seiki, M., Noda, T., and Shibuya, M. 2001. Involvement of Flt-1 tyrosine kinase (vascular endothelial growth factor receptor-1) in pathological angiogenesis. Cancer Res 61:1207-1213.
22. Sondell, M., and Kanje, M. 2001. Postnatal expression of VEGF and its receptor flk-1 in peripheral ganglia. Neuroreport 12:105-108.
23. Witmer, A. N., Blaauwgeers, H. G., Weich, H. A., Alitalo, K., Vrensen, G. F., and Schlingemann, R. O. 2002. Altered expression patterns of VEGF receptors in human diabetic retina and in experimental VEGF-induced retinopathy in monkey. Invest Ophthalmol Vis Sci 43:849-857.
24. Yang, K., and Cepko, C. L. 1996. Flk-1, a receptor for vascular endothelial growth factor (VEGF), is expressed by retinal progenitor cells. J Neurosci 16:6089-6099.
25. Ogata, N., Yamanaka, R., Yamamoto, C., Miyashiro, M., Kimoto, T., Takahashi, K., Maruyama, K., and Uyama, M. 1998. Expression of vascular endothelial growth factor and its receptor, KDR, following retinal ischemia-reperfusion injury in the rat. Curr Eye Res 17:1087-1096.
26. Suzuma, K., Takagi, H., Otani, A., Suzuma, I., and Honda, Y. 1998. Increased expression of KDR/Flk-1 (VEGFR-2) in murine model of ischemia-induced retinal neovascularization. Microvasc Res 56:183-191.

We claim:

1. A method of treating or preventing retinopathy in an individual in need thereof, comprising administering to said individual an effective amount of an agonist of VEGFR-1.

2. The method of claim 1, wherein the agonist is a growth factor selected from the group consisting of PIGF-1, PIGF-2, VEGF-A, and VEGF-B or an analog thereof.

3. The method of claim 1, wherein the retinopathy is retinopathy of prematurity.

4. A method of treating or preventing a disorder associated with blood vessel loss in an individual in need thereof, comprising administering to said individual an effective amount of an agonist of VEGFR-1.

5. The method of claim 4, wherein the agonist is a growth factor selected from the group consisting of PIGF-1, PIGF-2, VEGF-A, and VEGF-B or an analog thereof.

6. The method of claim 4, wherein the disorder associated with blood vessel loss is diabetic neuropathy.

7. The method of claim 6, wherein the agonist is a growth factor selected from the group consisting of PIGF-1, PIGF-2, VEGF-A, and VEGF-B or an analog thereof.


SEQUENCE LISTING (6 sequences)

SEQ ID NO: 1 - DNA, 25 bp, Artificial Sequence
  Description of Artificial Sequence Synthetic primer
gaggaggatgagggtgtctataggt

SEQ ID NO: 2 - DNA, 23 bp, Artificial Sequence
  Description of Artificial Sequence Synthetic primer
gtgatcagctccaggtttgactt

SEQ ID NO: 3 - DNA, 22 bp, Artificial Sequence
  Description of Artificial Sequence Synthetic primer
gccctgcctgtggtctcactac

SEQ ID NO: 4 - DNA, 20 bp, Artificial Sequence
  Description of Artificial Sequence Synthetic primer
caaagcattgcccattcgat

SEQ ID NO: 5 - DNA, 19 bp, Artificial Sequence
  Description of Artificial Sequence Synthetic primer
cagacgccactgtcgcttt

SEQ ID NO: 6 - DNA, 23 bp, Artificial Sequence
  Description of Artificial Sequence Synthetic primer
tgtctttggaactttgtctgcaa